(19) United States
(12) Patent Application Publication
(10) Pub. No.: US 2023/0127407 A1
(43) Pub. Date: Apr. 27, 2023

(54) INTRAOCULAR LENS REPOSITIONING DEVICE

(71) Applicant: University of South Florida, Tampa, FL (US)

(72) Inventor: Jason M. So, Tampa, FL (US)

(21) Appl. No.: 17/913,253

(22) PCT Filed: Mar. 25, 2021
(86) PCT No.: PCT/US2021/023931

§ 371 Date: Sep. 21, 2022

Related U.S. Application Data
(60) Provisional application No. 62/994,450, filed on Mar. 25, 2020.

(51) Int. Cl. A61F 2/16

(52) U.S. Cl. CPC A61F 2/1662; A61F 2002/1681

(57) ABSTRACT

An intraocular optic capture device is provided to replace an intraocular lens dislocated within an eye. The intraocular optic capture device includes a body defining an opening configured to receive and hold the intraocular lens. The intraocular optic capture device includes one or more fixing arms extending from the body and configured to fix the body to a structure of the eye.

### CROSS-REFERENCE TO RELATED APPLICATION

This application claims priority to U.S. Provisional Patent Application No. 62/994,450, filed on Mar. 25, 2020. The content of this application is incorporated by reference in its entirety.

### TECHNICAL FIELD

This document describes devices, systems, and methods related to devices, systems and methods for replacing an intraocular lens.

### BACKGROUND

Intraocular lens (IOL) is a lens implanted in the eye as part of a treatment for cataracts, myopia, or other illnesses. Some example IOLs includes a pseudophakic IOL. The pseudophakic IOL is implanted during cataract surgery after the natural lens (e.g., a cataract) has been removed from the eye. The IOL may provide the same light focusing function as the natural crystalline lens. Other example IOLs include a phakic intraocular lens (PIOL). The PIOL is a lens that is placed over the existing natural lens and is used in refractive surgery to change the eye's optical power as a treatment for myopia. The implanted IOL may carry several risks associated with eye surgeries. For example, the IOL can be dislocated after surgery.

### SUMMARY

A technique described herein provides a solution to replace a dislocated intraocular lens (IOL), such as a dislocated single-piece IOL. Some implementations of the technique employs an intraocular optic capture device (IOCD) configured to reposition the dislocated IOL in an eye. The IOCD can include an opening for capturing the dislocated IOL. In some implementations, the IOCD can be configured as a ring with the opening. The IOCD can be made of a flexible material, such as acrylic, which allows optic capture of the dislocated IOL.

In some implementations, the IOCD can be used to reposition the dislocated IOL in the posterior chamber without need to create a large corneal or scleral incision. Some example procedures can include enabling access to the posterior segment in the eye, and injecting the IOCD in the anterior chamber of the eye, repositioning the dislocated IOL in the anterior chamber, capturing the dislocated IOL with the opening of the IOCD, and fixing the IOCD to a structure within the eye. For example, a surgical method, such as vitrectomy, can be used to remove some or all of the vitreous humor from the system to allow access to the dislocated lens in the posterior segment of the eye. Alternatively, pars plana vitrectomy (PPV) can be used for such a surgical method. PPV is a technique in vitreoretinal surgery that enables access to the posterior segment for treating conditions such as retinal detachments, vitreous hemorrhage, endophthalmitis, and macular holes in a controlled, closed system.

In some implementations, the IOCD includes one or more fixing arms (e.g., haptics), and the IOCD that captures the dislocated IOL can be scleral-fixated using sutures that are fed through the fixing arms of the IOCD. The fixing arms can be configured in various configurations. In some implementations, the IOCD can include one or more suture holes as the fixing arms, which are arranged around the ring of the IOCD and configured to receive sutures therethrough. For example, the IOCD can include four suture holes, two of which are arranged at one side of the ring of the IOCD and the other two of which are arranged at the opposite side of the ring of the IOCD. In alternative implementations, the IOCD can include two extended arms or haptics configured as strands, one of which extends from one side of the ring of the IOCD and the other of which extends from the opposite side of the ring of the IOCD. For example, the extended arms or haptics may then be scleral-fixated through a tunneled incision in the sclera 180 degrees apart.

The IOCD may be used to fixate a dislocated lens, instead of removing the dislocated lens from the eye. For example, dislocated lenses that offer astigmatic (refractive error in certain axis) correction or presbyopia (age-related loss of near-sightedness) correction are typically extracted from the eye and not reused. Instead, the IOCD described herein can be used to fixate such a dislocated lens so that the lens can be positioned in a certain axis and will not rotate. Thus, ophthalmologists can use the IOCD to fixate astigmatic correction lenses (known as “Toric lenses”) and presbyopia correction lenses (known as “premium lenses” or “extended depth-of-focus lenses”), without having to remove them from the eye.

Particular embodiments described herein include an intraocular optic capture device for repositioning an implanted intraocular lens in an eye. The device may include a body and one or more fixing arms. The body may define an opening configured to receive and hold at least a portion of the intraocular lens. The fixing arms may extend from the body and be configured to fix the body to a structure of the eye.

In some implementations, the system can optionally include one or more of the following features. The opening may be sized to be identical a lens portion of the intraocular lens. Alternatively, the opening may be sized to be smaller than a size of a lens portion of the intraocular lens. The opening may be configured to interference fit with a lens portion of the intraocular lens. The opening may be circular. Each of the fixing arms may define a suture opening configured to receive a suture for fixing the body to the structure of the eye. The structure of the eye may include an eye wall or a sclera. The fixing arms may be symmetrically arranged relative to a center of the opening. The fixing arms may be arranged in a same plane as a plane in which the body is placed. The fixing arms may be arranged in a different plane from a plane in which the body is placed. The fixing arms may include one or more strands. The strands of the fixing arms may include hooked portions. The strands may be made of polyvinylidene fluoride (PVDF) monofilament. The body may be made of a flexible material. The body may be made of acrylic. The intraocular lens may be an astigmatic correction lens or a presbyopia correction lens. The intraocular optic capture device may be configured to position the intraocular lens in a predetermined axis and prevent rotation of the intraocular lens.

Particular embodiments described herein include a method for repositioning an intraocular lens dislocated in an eye. The method may include inserting an intraocular optic capture device in an eye chamber; moving the intraocular optic capture device onto the intraocular lens; capturing a lens portion of the intraocular lens with the intraocular optic capture device; and fixing the intraocular optic capture device to a structure of the eye.

In some implementations, the system can optionally include one or more of the following features. The capturing the lens portion may include placing an opening of the intraocular optic capture device onto the lens portion of the intraocular lens. The lens portion of the intraocular lens may be at least partially inserted through the opening of the intraocular optic capture device. The capturing the lens portion may include interference-fitting the opening of the intraocular optic capture device with the lens portion of the intraocular lens. The fixing the intraocular optic capture device may include routing one or more sutures through one or more suture holes of the intraocular optic capture device; and stitching the one or more sutures with the structure of the eye. The one or more suture holes may be arranged symmetrically relative to a center of the intraocular optic capture device. The intraocular lens may be an astigmatic correction lens or a presbyopia correction lens. The intraocular optic capture device may be configured to position the intraocular lens in a predetermined axis and prevent rotation of the intraocular lens.

The details of one or more implementations are set forth in the accompanying drawings and the description below. Other features and advantages will be apparent from the description and drawings, and from the claims.

### BRIEF DESCRIPTION OF THE DRAWINGS

FIG. 1 illustrates an example system for replacing an intraocular lens within an eye.

FIGS. 2A-B illustrate example placement of an intraocular lens in an eye.

FIG. 3 illustrates another example intraocular lens.

FIG. 4 is a flowchart of an example process for replacing a dislocated intraocular lens with an intraocular optic capture device.

FIGS. 5A-B illustrate an example of captured intraocular lens within an intraocular optic capture device.

FIG. 6 illustrates an example intraocular optic capture device.

FIG. 7 illustrates another example intraocular optic capture device.

FIG. 8A is a top view of yet another example intraocular optic capture device.

FIG. 8B is a front view of the intraocular optic capture device of FIG. 8A.

FIG. 9A illustrates yet another example intraocular optic capture device.

FIG. 9B is a side view of the intraocular optic capture device of FIG. 9A.

### DETAILED DESCRIPTION OF ILLUSTRATIVE EMBODIMENTS

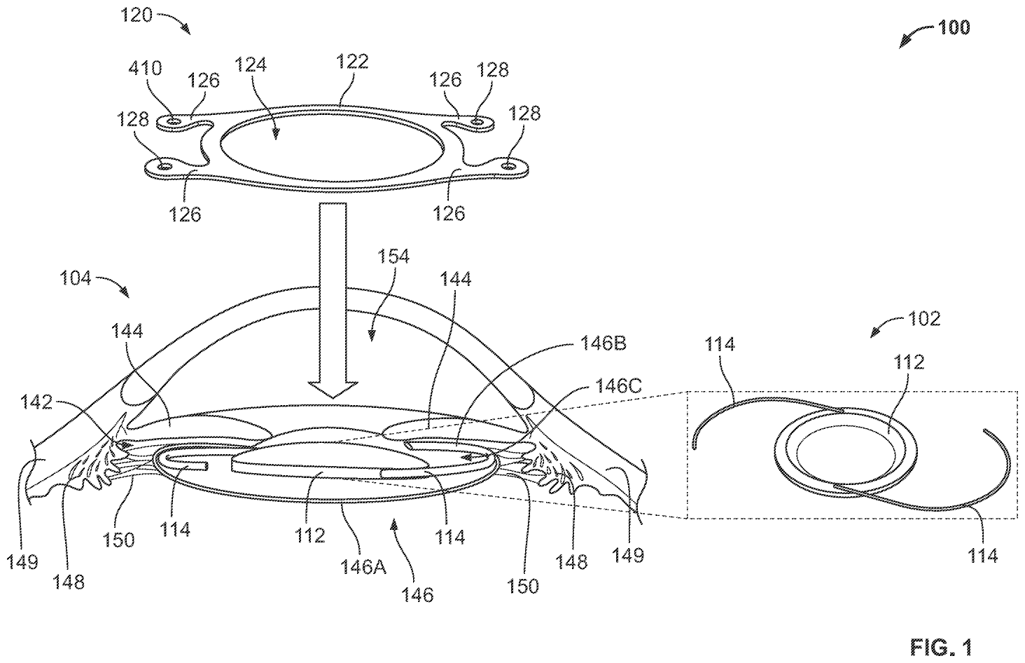

FIG. 1 illustrates a system 100 for replacing an implanted intraocular lens (IOL) 102 that is dislocated within an eye 104. The IOL 102 is a lens implanted in the eye as part of a treatment for cataracts, near-sighted eyes (myopia), far-sighted eyes, astigmatic eyes, or other illnesses. Various types of the IOL can be used for such treatment. One example type of the IOL 102 is a pseudophakic IOL. The pseudophakic IOL can be implanted during cataract surgery, after the cataract is removed. For example, the crystalline lens is first extracted and an IOL replaces it in a process that is very similar to cataract surgery. The pseudophakic IOL can be configured to provide the same light focusing function as the natural crystalline lens. Another example type of the IOL 102 is a phakic intraocular lens (PIOL) that is implanted without removing the patient's natural crystalline lens. The PIOL is a lens that is placed over the existing natural lens and is used in refractive surgery to change the eye's optical power as a treatment for myopia.

Referring to FIG. 1, the IOL 102 can include a lens portion 112 with haptics 114. The lens portion 112 can be made of plastic and configured to replace the natural eye lens (e.g., cataract). The haptics 114 are configured to hold the lens portion 112 in place in the capsular bag inside the eye 104.

The IOL 102 can be made of various materials. In some implementations, the IOL 102 can be made of an inflexible material, such as polymethylmethacrylate (PMMA). Alternatively, the IOL 102 can be made of flexible materials, such as silicone, acrylic glass, hydrophobic acrylate, hydrophilic acrylate, collamer, and other suitable materials. The IOL 102 can include soft foldable inert materials, such as silicone, acrylic glass, etc. Such materials can allow the lens to be folded and inserted into the eye through a smaller incision. For example, acrylic lenses can be used for patients who have a history of uveitis or are likely to have to undergo retinal surgery requiring vitrectomy with replacement by silicone oil, such as persons with proliferative diabetic retinopathy or who are at high risk of retinal detachment, such as persons with high myopia.

The lens portion 112 of the IOL 102 can include a monofocal lenses matched to distance vision. Alternatively, the lens portion 112 can be a multifocal lens to provide the patient with multiple-focused vision at far and reading distance. Alternatively, the lens portion 112 can be an adaptive lens provides the patient with limited visual accommodation.

In some implementations, the IOL 102 can be implanted under local anesthesia with the patient awake throughout the operation. A flexible IOL enables the lens to be rolled for insertion into the capsule through a very small incision, thus avoiding the need for stitches. This procedure can be completed in a short period of time, such as less than 30 minutes. The recovery period is about 2-3 weeks.

The IOL 102 can be implanted in the posterior chamber of the eye 104. Alternatively, the IOL 102 can be implanted in the anterior chamber. Various forms of surgery may be used to remove natural crystalline lenses (e.g., cataracts), such as extracapsular cataract extraction, intracapsular extraction, etc. For example, after a natural crystalline lens (e.g., a cataractous lens) is extracted, an intraocular lens (IOL) can be implanted in either the anterior or the posterior chamber of the eye. In an anterior chamber implant, the IOL can be situated forward of, or mounted to the iris. In a posterior chamber implant, the IOL can be situated behind the iris and may be mounted within the cleft or fornix of the capsule which remains in place after extracapsular surgery.

In either anterior or posterior chamber implant, the IOL may be centered and fixed in position by one or more supporting strands or haptic members (e.g., the haptics 114). The haptic members of an IOL can have various geometric shapes and configurations. The haptic members can be flexible strands of non-biodegradable material which is fixed to the lens body. The haptic members can have spring-like memory qualities so that the haptic members can be compressed or offset from the normal rest position and thereafter returned to the fully extended condition when pressure is removed.

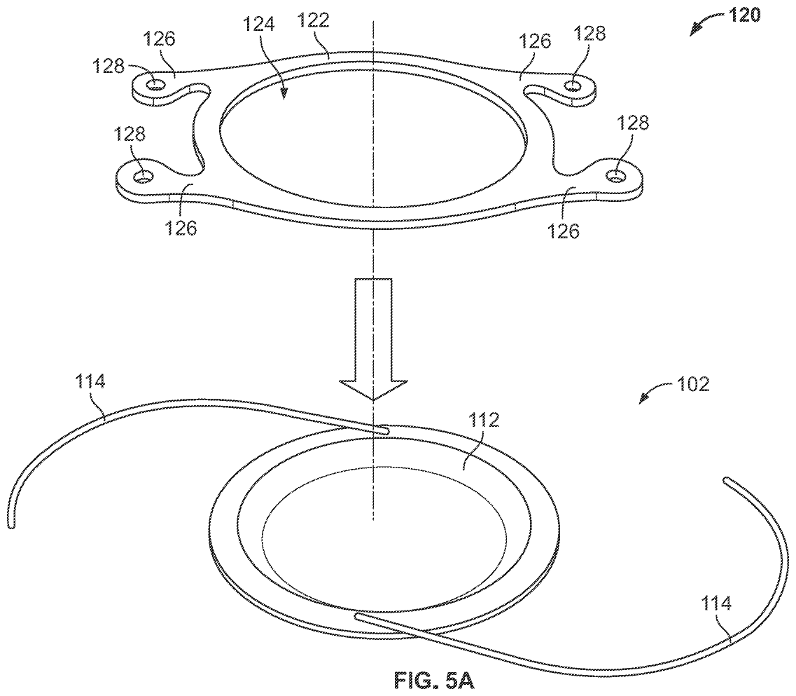

Referring to FIG. 1, the IOL 102 is implanted in a posterior chamber 142 of the eye 104, the lens portion 112 can be seated against a posterior capsule 146 and creates a space between the capsule 146 and the lens portion 112. In some implementations, the IOL 102 can be implanted in the posterior chamber 142 behind an iris 144. For example, the IOL 102 can be a 3-piece IOL as depicted in FIGS. 1 and 5A, which may include two curved haptics attached to a lens portion 180 degree apart. The natural lens (e.g., cataract) has been extracted from the capsular bag 146 while leaving a posterior wall 146A and an annular flap portion 146B forming a cleft or fornix 146C. The capsular bag 146 is connected to a ciliary muscle in an eye wall 148 via suspensory ligaments 150. Vitreous humor in the region behind the capsular bag 146 can be prevented from flowing forward by the posterior wall 146A that assumes a generally planar shape.

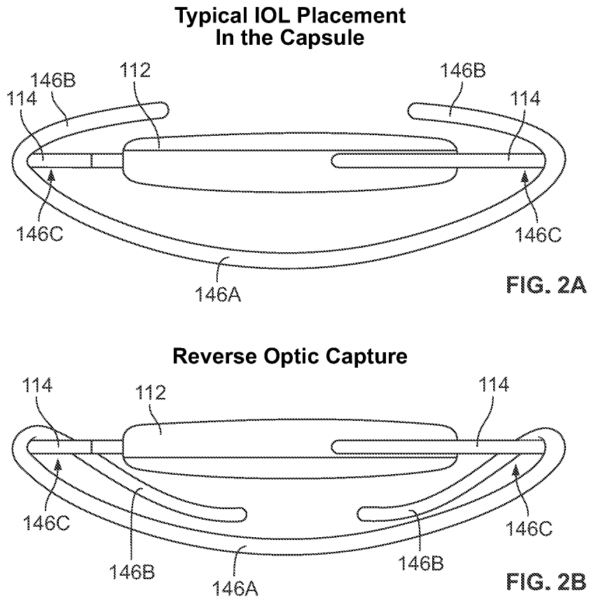

In some implementations, when the IOL 102 is implanted in the posterior chamber of the eye, the haptics 114 can support the lens portion 112 by engaging the cleft or fornix portion 146C, thereby fixing the lens portion 112 in the eye 104. For example, as depicted in FIGS. 1 and 2A, the haptics 114 can engage with the cleft or fornix portion 146C with the annular flap portion 146B extending over one side of the IOL 102 opposite to the posterior wall 146A. Alternatively, as illustrated in FIG. 2B, the annular flap portion 146B can be routed around the haptics 114 and run between the lens portion 112 of the IOL 102 and the posterior wall 146A.

In other implementations, the IOL 102 can be adapted to be implanted in an anterior chamber 154 of the eye 104, which is anterior to the iris 144 of the eye 104. For example, the IOL 120 may be a special version of intraocular lens that is referred to as an anterior chamber IOL (ACIOL).

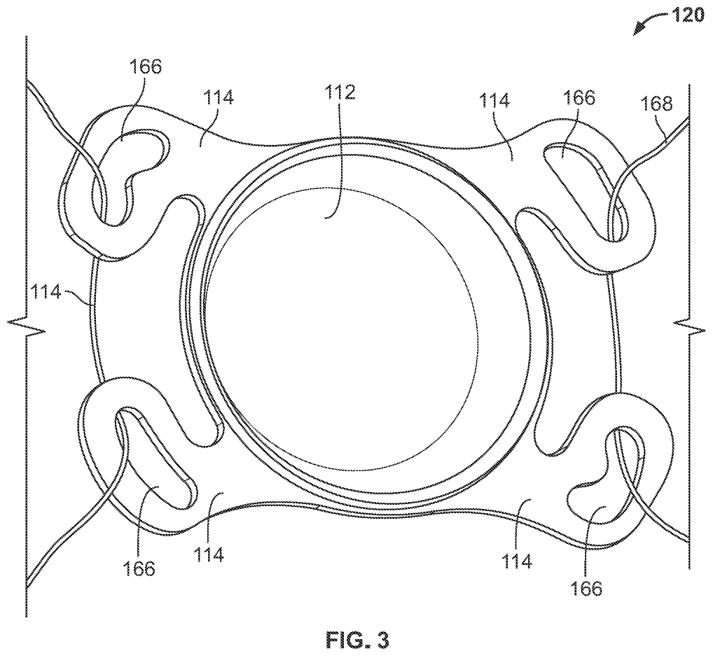

FIG. 3 illustrates another example intraocular lens (IOL) 102. In this example, the IOL 102 includes a lens portion 112 with one or more haptics 114 extending from the lens portion 112. In the illustrated example, the IOL 102 includes four haptics 114 that are symmetrically positioned relative to the lens portion 112. The haptics 114 can include suture holes 166 each configured to route a suture 168 therethrough for fixation of the IOL 102 to an eye structure (e.g., the eye wall 148 and/or a sclera 149 of FIG. 1).

The implanted IOL 102 can carry several risks associated with eye surgeries, such as infection, dislocation of the lens (e.g., loosening of the lens, lens rotation, etc.), inflammation, or nighttime halos.

Referring to FIG. 1, the system 100 can include an intraocular optic capture device (IOCD) 120 configured to replace the IOL 102 that is dislocated in the eye 104. The IOCD 120 can include a body 122 and one or more fixing arms 126. The body 122 defines an opening 124 configured to capture the lens portion 112 of the dislocated IOL 102 in the eye 104. For example, the IOCD 120 can be placed onto or over the existing IOL 102 in the eye 104 such that the opening 124 of the body 122 is arranged to surround the lens portion 112 of the IOL 102 and thus hold the IOL 102 in place.

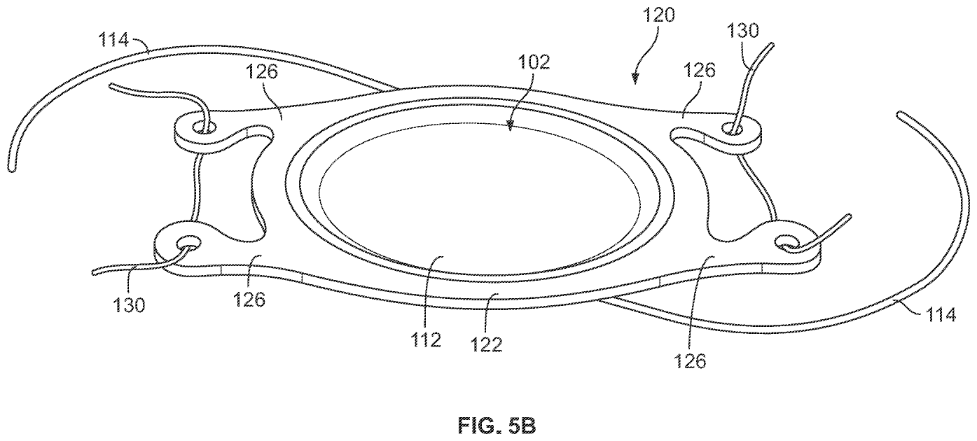

The fixing arms 126, which may also referred to herein as haptics, are configured and used to fix the body 122 of the IOCD 120 to a suitable structure in the eye 104 when the body 122 captures the lens portion 112 of the dislocated IOL 102. For example, the fixing arms 126 extend from the body 122 of the IOCD 120 and provide features to fix the body 122 of the IOCD 120 to an eye structure. In the illustrated example, the fixing arms 126 include suture holes 128 configured to receive sutures which are fastened to a suitable eye structure adjacent to the IOL 102 and/or the IOCD 120. Various implementations of the IOCD 120 are described in more detail herein, for example with reference to FIGS. 6-9.

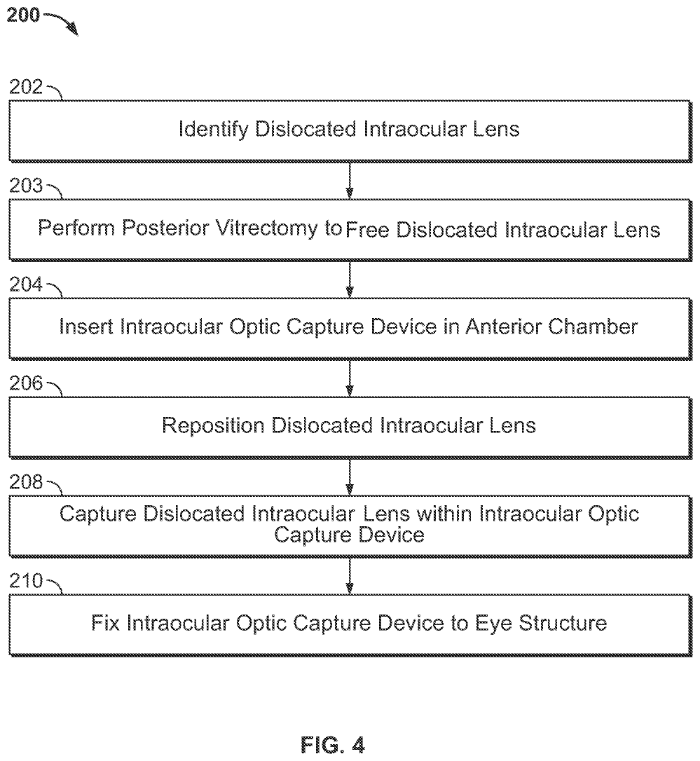

FIG. 4 is a flowchart of an example process 200 for replacing a dislocated IOL with an IOCD. The process 200 can include identifying a dislocated IOL (e.g., the IOL 102) (202). The dislocated IOL can be identified from an image of the eye, such as a digital image of the IOL and features around the IOL, that is captured by an image capture device. Alternatively or in addition, the dislocated IOL can be identified by a practitioner, such as an ophthalmologist, with or without assistance of an image capture device, before or during a surgical operation.

The process 200 can include performing posterior vitrectomy to free the dislocated intraocular lens (203). Vitrectomy or other suitable surgical methods can be used to remove some or all of the vitreous humor to allow access to the dislocated lens in the posterior segment of the eye.

The process 200 can include inserting an IOCD in an eye chamber (204). For example, a tiny incision can be created to gain access to an eye chamber, and the IOCD is inserted through the incision into the eye chamber. In some implementations, the IOCD is repositioned and fixated in the eye chamber.

The process 200 can include repositioning the dislocated IOL in the eye chamber (206). For example, the dislocated IOL is moved and located to a desired location in the eye chamber (e.g., the anterior chamber). By way of example, the practitioner (e.g., the ophthalmologist) can use an instrument to engage with or grab a portion of the IOL and reposition the IOL in the eye chamber.

The process 200 can include capturing the IOL with the IOCD (208). For example, the IOCD can be placed onto the IOL that has been repositioned to a desired location, such that an opening of the IOCD (e.g., the opening 124 of the IOCD 120) captures at least part of a lens portion of the IOL (e.g., the lens portion 112). Thus, the lens portion of the IOL can be fitted into the opening of the IOCD, so that the IOL is held in place by the IOCD.

The process 200 can include fixing the IOCD to a suitable structure in the eye (210). The IOCD that captures the IOL can be fixedly placed within the eye, and thus replace the IOL in a desired location (or the original location of the IOL) within the eye. Various methods can be used to fix the IOCD to an eye structure. In some implementations, the IOCD can be fastened to the eye structure using sutures, strands, wires, or other stitching elements. For example, sutures can be routed through one or more holes included in the IOCD and stitched to a desired eye structure to fix the IOCD to the eye structure. In other implementations, the IOCD can include one or more hooked or bendable arms that can be hooked or inserted in, or otherwise held by, a suitable eye structure.

Referring to FIGS. 5A-B, an example structure and process for capturing the IOL 102 with the IOCD 120. As described with reference to FIG. 1, the IOCD 120 includes the body 122 and the fixing arms 126. The opening 124 of the body 122 is configured and sized to fit the lens portion 112 of the IOL 102, thereby rearranging the lens portion 112 of the IOL 102 in place within the eye. As illustrated in FIG. 5A, the IOCD 120 can be positioned above the IOL 102 such that the opening 124 of the IOCD 120 is aligned with the lens portion 112 of the IOL 102. Then, the IOCD 120 is moved onto the IOL 102 until the body 122 of the IOCD 120 is placed over the lens portion 112 of the IOL 102. As illustrated in FIG. 5B, the lens portion 112 can be interference-fit (e.g., snap-fit) into the opening 124 of the IOCD 120 so that the IOL 102 is held by the IOCD 120. For example, as described herein, the body 122 of the IOCD 120 can be made of a flexible material which permits for the body 122 of the IOCD 120 to fully engage with the circumference of the IOL such that the lens portion 112 of the IOL 102 is at least partially inserted to the opening 124 of the IOL 102.

The fixing arms 126 of the IOCD 120 can be engaged with a structure of the eye to fixedly support the IOCD 120 holding the IOL 102 within the eye. In the illustrated example, the fixing arms 126 of the IOCD 120 include the suture holes 128 through which sutures 130 are routed. The sutures 130 can be stitched to a structure of the eye, such as the eye wall 148 and/or the sclera 149 in FIG. 1. Various types of sutures 130 can be used for fixation of the IOCD 120, such as scleral fixation. Examples of the sutures 130 include microporous, monofilament sutures of flexible biomaterial, such as Gore-Tex® sutures (e.g., 8-0 Gore-Tex® sutures) or monofilament polypropylene sutures.

Referring to FIGS. 6-9, various implementations of the IOCD are described. FIG. 6 illustrates an example of the IOCD 120. As described herein, the IOCD 120 includes the body 122 that defines the opening 124. The body 122 provides an outer edge of the opening 124. The opening 124 of the IOCD 120 can be configured and sized to correspond to the lens portion 112 of the IOL 102 that is captured by the IOCD 120. In embodiments where the lens portion 112 of the IOL 102 is circular, the opening 124 of the IOCD 120 can be configured to be circular. The opening 124 of the IOCD 120 can be sized to be identical or similar to the size of the lens portion 112. In some implementations, the opening 124 of the IOCD 120 can have a diameter that is slightly smaller than a diameter of the lens portion 112 of the IOL 102 so that the lens portion 112 is snap fit with the opening 124 of the IOCD 120. In some implementations, the opening 124 of the IOCD 120 can have a diameter D₁ that ranges between 5 mm and 5.5 mm. A smaller or larger diameter of the opening 124 are possible based at least part on the diameter of the lens portion 112 of the IOL 102 that is held by the IOCD 120. An outer diameter D_{O} of the body 122 can range between 5.5 mm and 6 mm. The outer diameter D_{O} of the body 122 can be smaller or larger based at least part on the size of the opening 124. The body 122 of the IOCD 120 can have a thickness D_{T} that ranges between 0.3 mm and 1.5 mm, and preferably around 1.0 mm. Other thicknesses D_{T} of the body 122 are also possible based at least part on the size of the opening 124, the size of the body 122, the lens portion 112 of the IOL 102, or other dimensions of the IOL 102.

The body 122 of the IOCD 120 can be made of a flexible material. Examples of such a flexible material include silicone, acrylic glass, hydrophobic acrylate, hydrophilic acrylate, collamer, and other suitable flexible materials. In some implementations, the body 122 of the IOCD 120 can be made of the same material as the lens portion 112 of the IOL 102. In other implementations, the body 122 of the IOCD 120 can be made of a different material from the lens portion 112 of the IOL 102. In addition, the body 122 of the IOCD 120 can be made of a sterile material.

Referring to FIG. 6, the IOCD 120 can include one or more fixing arms 126. In this example, the IOCD 120 includes four fixing arms 126 extending from the body 122. The fixing arms 126 can be arranged symmetrically about a center C of the opening 124 so that the IOCD 120 is balanced when fixed using the fixing arms 126. For example, two fixing arms 126A-B are arranged at one side of the body 122 (e.g., a left side of a first (e.g., vertical) body axis A_{V} extending through the center C) while the other two fixing arms 126C-D are arranged at the opposite side of the body 122 (e.g., a right side of the first body axis A_{V}).

The fixing arm 126 includes a neck portion 132 and a distal portion 134. The neck portion 132 extends between the body 122 and the distal portion 134. The distal portion 134 includes a suture hole 128. The neck portion 132 can have a width W_{N} smaller than an outer width or diameter W_{D} of the distal portion 134. In other implementations, the width W_{N} of the neck portion 132 can be identical to or larger than the outer width or diameter W_{D} of the distal portion 134.

The fixing arms 126 includes the suture holes 128 at distal ends (e.g., in the distal portions 134). The suture holes 128 can be arranged symmetrically about the center C of the opening 124 to provide balance of the IOCD 120 when fixed using sutures routing through the suture holes 128. The suture holes 128 can be arranged at an angle (ANG1, ANG2, ANG3, and ANG4) relative to the first body axis A_{V} (or a second (e.g. horizontal) body axis A_{H} extending through the center C and perpendicular the first body axis A_{V}). In some implementations, the suture holes 128 can be arranged at the same angle relative to the first body axis A_{V}, which can range between 10 degrees and 80 degrees. In other implementations, at least one of the suture holes 128 can be arranged at a different angle relative to the first body axis A_{V}.

The fixing arms 126 can be made of the same material as the body 122. Alternatively, the fixing arms 126 can be made of a different material from the body 122. The fixing arms 126 can be made to be a single piece with the body 122. Alternatively, the fixing arms 126 can be made separately from the body 122 and connected to the body 122 in various ways, such as using adhesive and/or fasteners of various types. The fixing arms 126 can have the same thickness as the thickness D_{T} of the body 122. Alternatively, the fixing arms 126 can have a smaller or larger thickness than the thickness D_{T} of the body 122.

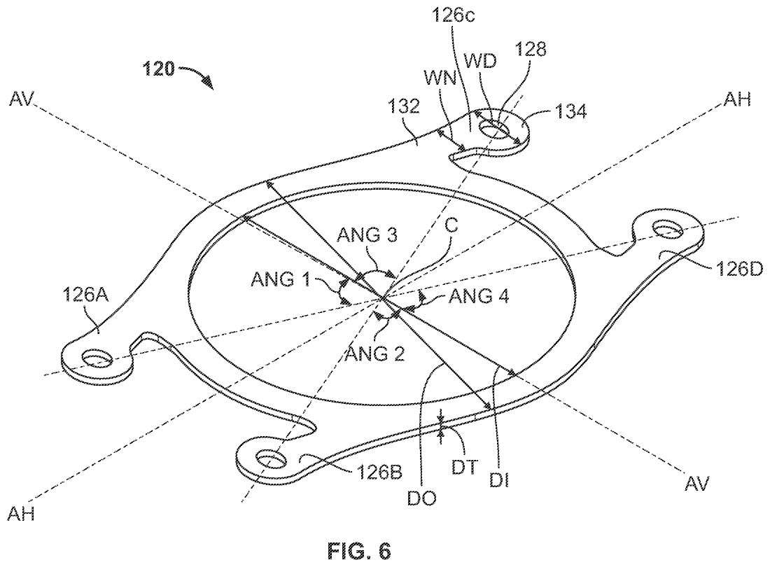

FIG. 7 illustrates another example of the IOCD 120. The IOCD 120 in this example is configured similarly to the IOCD of FIG. 6 except for the configurations of the fixing arms 126. In the example of FIG. 7, the fixing arm 126 is connected to the body 122 at a proximate end 136 and extends to a distal end 138. The fixing arm 126 can be configured to have a width W_{D} that gradually decreases from the proximate end 136 towards the distal end 138 so that the largest width of the fixing arm 126 is connected to the body 122.

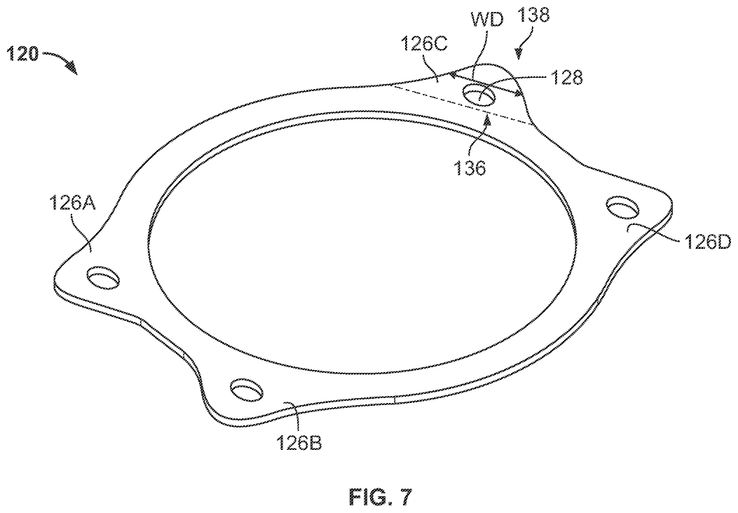

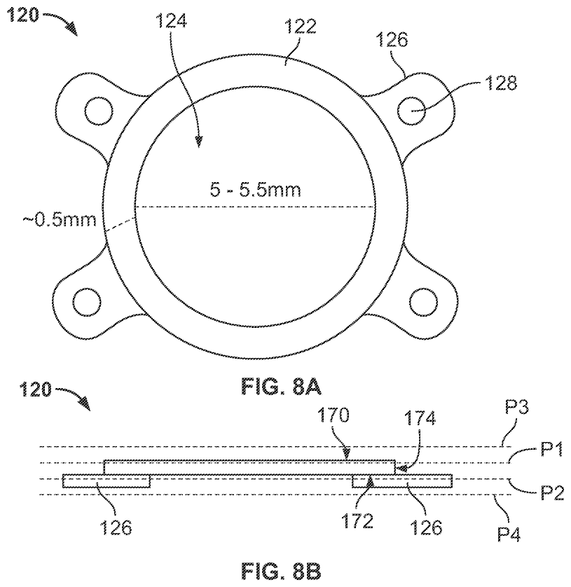

FIGS. 8A-B illustrates yet another example of the IOCD 120. The IOCD 120 in this example is configured similarly to the IOCD of FIG. 6 or FIG. 7 except for the configurations of the fixing arms 126. In the example of FIGS. 8A-B, the fixing arms 126 are arranged in a different plane from the body 122. As shown in FIG. 8B, the body 122 is arranged in a first plane P1 and the fixing arms 126 are arranged in a second plane P2 that is different from the first plane P1. The second plane P2 can be positioned in parallel with the first plane P1. Alternatively, the second plane P2 can be angled from the first plane P1.

The body 122 has opposite main surfaces 170, 172 that are connected to a lateral surface 174. The fixing arm 126 can be connected to one of the main surfaces 170, 172 of the body 122 and extend out in a direction perpendicular to the lateral surface 174 of the body 122. In some implementations, all of the fixing arms 126 can be arranged in the same plane (e.g., the second plane P2) while the body 122 is arranged in another plane (e.g., the first plane P1). In other implementations, at least one of the fixing arms 126 can be arranged in a plane different than the other fixing arms. For example, at least one of the fixing arms 126 can be arranged in the same plane (e.g., the first plane P1) as the body 122. Alternatively or in addition, at least one of the fixing arms 126 can be arranged in a plane (e.g., a third plane P3) opposite to the second plane P2 relative to the first plane P3. Alternatively or in addition, at least one of the fixing arms 126 can be arranged in a plane (e.g., a fourth plane P4) at the same side of the body 122 but further away from the second plane P2.

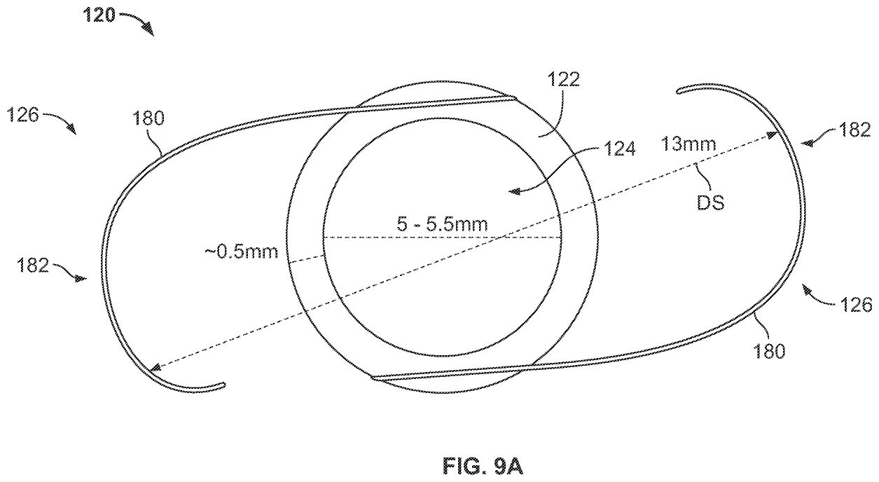

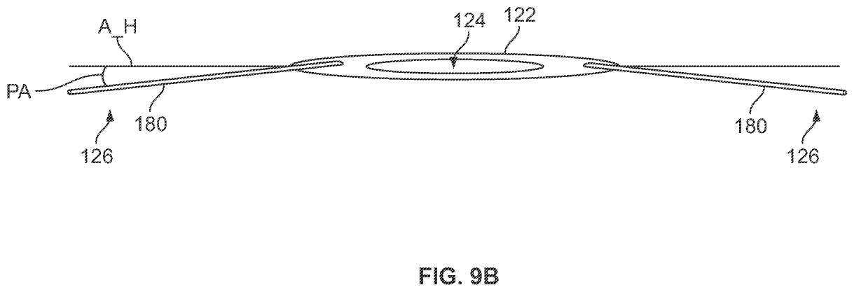

FIGS. 9A-B illustrate yet another example of the IOCD 120. The IOCD 120 in this example is configured similarly to the IOCD of FIG. 6, FIG. 7, or FIGS. 8A-B except for the configurations of the fixing arms 126. In this example, the fixing arms 126 are configured as strands or wires 180. In the example, the IOCD 120 includes two strands 180 fixed to the body 122 and extending therefrom. In alternative examples, the IOCD 120 can include a single strand or three or more strands.

The strands 180 can be made to be flexible. The strands 180 can have spring-like memory qualities so that the haptic members can be compressed or offset from the normal rest position and thereafter returned to the fully extended condition when pressure is removed. For example, the strands 180 can have hooked portions 182 configured to hook in a structure of the eye (e.g., the eye wall 148 and/or the sclera 149 in FIG. 1) or hung between structures of the eye (e.g., the cleft 146C in FIG. 1). Alternatively, the strands 180 can be bendable and shapeable so that the strands can be bent to a shape and hold the shape.

In the illustrated example of FIG. 9, each of two strands 180 extend tangentially from the body 122 along a substantially circular arc exterior to the body 122. The strands 180 can be arranged symmetrically relative to the center C of the opening 124. For example, the strands 180 can be spaced apart at a predetermined distance D_{S} (between corresponding features of the strands 180) along a line passing through the center C of the opening 124. The distance can range between 8 mm and 16 mm, and preferably about 13 mm. The strands 180 can be arranged at opposite sides of the body 122 at a predetermined angle relative to a plane that is parallel to the body 122. For example, as illustrated in FIG. 9B, the strand 180 can be displaced posteriorly at an angle PA relative to a plane or axis A_{H} that is parallel to the body 122. The angle PA can range from 1 degree to 10 degree, and preferably around 5 degree.

The strands 180 can have a circular cross section. Alternatively, other geometric shapes can be used for the strands 180, such as rectangular, square, or oval. In some implementations, the strands 180 can be made of polyvinylidene fluoride (PVDF) monofilament. Other materials are also possible for the strands 180.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

1. An intraocular optic capture device for repositioning an implanted intraocular lens in an eye, the device comprising:
a body defining an opening configured to receive and hold at least a portion of the intraocular lens; and
one or more fixing arms extending from the body and configured to fix the body to a structure of the eye.

2. The intraocular optic capture device of claim 1, wherein the opening is sized to be identical a lens portion of the intraocular lens.

3. The intraocular optic capture device of claim 1, wherein the opening is sized to be smaller than a size of a lens portion of the intraocular lens.

4. The intraocular optic capture device of claim 3, wherein the opening is configured to interference fit with a lens portion of the intraocular lens.

5. (canceled)

6. The intraocular optic capture device of claim 1, wherein each of the fixing arms defines a suture opening configured to receive a suture for fixing the body to the structure of the eye.

7. The intraocular optic capture device of claim 6, wherein the structure of the eye includes an eye wall or a sclera.

8. The intraocular optic capture device of claim 1, wherein the fixing arms are symmetrically arranged relative to a center of the opening.

9. The intraocular optic capture device of claim 1, wherein the fixing arms are arranged in a same plane as a plane in which the body is placed.

10. The intraocular optic capture device of claim 1, wherein the fixing arms are arranged in a different plane from a plane in which the body is placed.

11. The intraocular optic capture device of claim 1, wherein the fixing arms include one or more strands.

12. The intraocular optic capture device of claim 11, wherein the strands of the fixing arms include hooked portions.

13. The intraocular optic capture device of claim 11, wherein the strands are made of polyvinylidene fluoride (PVDF) monofilament.

14. The intraocular optic capture device of claim 1, wherein the body is made of a flexible material.

15. The intraocular optic capture device of claim 1, wherein the body is made of acrylic.

16. A method for repositioning an intraocular lens dislocated in an eye, the method comprising:
inserting an intraocular optic capture device in an eye chamber;
moving the intraocular optic capture device onto the intraocular lens;
capturing a lens portion of the intraocular lens with the intraocular optic capture device; and
fixing the intraocular optic capture device to a structure of the eye.

17. The method of claim 16, wherein capturing the lens portion comprises:
placing an opening of the intraocular optic capture device onto the lens portion of the intraocular lens, wherein the lens portion of the intraocular lens is at least partially inserted through the opening of the intraocular optic capture device.

18. The method of claim 17, wherein capturing the lens portion further comprises:
interference-fitting the opening of the intraocular optic capture device with the lens portion of the intraocular lens.

19. The method of claim 16, wherein fixing the intraocular optic capture device comprises:
routing one or more sutures through one or more suture holes of the intraocular optic capture device; and
stitching the one or more sutures with the structure of the eye.

20. The method of claim 19, wherein the one or more suture holes are arranged symmetrically relative to a center of the intraocular optic capture device.

21. The intraocular optic capture device of claim 1, wherein the intraocular lens is an astigmatic correction lens or a presbyopia correction lens, and wherein the intraocular optic capture device is configured to position the intraocular lens in a predetermined axis and prevent rotation of the intraocular lens.

22. (canceled)
